(19) United States
(12) Patent Application Publication
(10) Pub. No.: US 2022/0193631 A1
(43) Pub. Date: Jun. 23, 2022

(54) MAGNESIUM OXIDE SORBENTS FOR ROOM TEMPERATURE CARBON DIOXIDE ADSORPTION AND METHODS FOR THEIR FABRICATION

(71) Applicant: Singapore University Of Technology And Design, Singapore (SG)

(72) Inventors: Ping Wu, Singapore (SG); Andrei LEBEDEV, SINGAPORE (SG); Franklin ANARIBA, SINGAPORE (SG); Victor CHEN YAOHUI, SINGAPORE (SG)

(21) Appl. No.: 17/139,510

(22) Filed: Dec. 31, 2020

Related U.S. Application Data
(60) Provisional application No. 63/128,584, filed on Dec. 21, 2020.

(51) Int. Cl. B01J 20/22; C01F 5/24; B01J 20/04; B01J 20/28; B01J 20/30; C07C 51/41; B01D 53/62; B01D 53/81

(52) U.S. Cl. CPC B01J 20/223; C01F 5/24; B01J 20/043; B01J 20/28023; B01J 20/3085; B01J 20/3078; B01D 2257/504; C07C 51/418; B01D 53/62; B01D 53/81; B01D 2253/1124; B01D 2253/25; B01J 20/3007

(57) ABSTRACT

Methods for fabrication of and use of magnesium oxide sorbents for room temperature carbon dioxide adsorption are provided. In accordance with one aspect, a method for fabrication of sorbents is provided which includes using calcination to obtain MgO—Mg(OH)₂ nano-composites and aging the MgO—Mg(OH)₂ nano-composites to form nano MCHs for room temperature carbon dioxide adsorption. According to another aspect, a method for fabrication of sorbents which includes fabrication of monoclinic magnesium malate tetrahydrate (C₈H₁₀MgO₁₀.4H₂O) and use of such sorbents for room temperature carbon dioxide adsorption is provided.

### TECHNICAL FIELD

The present invention generally relates to carbon dioxide adsorption materials, and more particularly relates to magnesium oxide sorbents for room temperature carbon dioxide adsorption and methods of their fabrication.

### BACKGROUND OF THE DISCLOSURE

Over the past decades, global energy demand has raised significantly with accelerated industrial and population growth, which is a threat to the environment. The continuous increase of anthropogenic carbon dioxide (CO₂) emissions from fossil fuel combustion in many industries has been identified as the major contributor to global warming, which is a pressing concern. Therefore, establishment of advanced technologies to reduce or control CO₂ emissions from industries is considered to be crucial. Inevitably, fossil fuels are predominant as an indispensable energy source.

While there have been significant improvements in utilizing alternative energy sources, it may take a long time to replace fossil fuels as the sole energy source. Accordingly, it is necessary to take measures to control the rate of CO₂ emission from industries and current CO₂ concentrations in the atmosphere. Significant efforts have been placed on developing efficient CO₂ capture, mineralizing, or storage technologies, which can reduce the CO₂ significantly in a short period of time. The adsorption method is a promising method for CO₂ capture as it possesses numerous advantages such as no liquid waste, a wide range operating temperature, and less energy requirements for regeneration. Utilizing solid adsorbents to capture CO₂ is considered an effective technique for subsequent CO₂ storage instead of direct emission.

Among numerous solid CO₂ adsorbents, magnesium oxide (MgO) is considered to be a promising candidate for CO₂ capture. MgO is an inexpensive, abundant, and non-toxic earth material that exhibits a wide operation temperature with promising theoretical capture capacities. However, the monetary cost and carbon footprint of extracting MgO is significantly higher than that of Mg(OH)₂. Nevertheless, Mg(OH)₂ can be extracted from abandoned brine produced in a desalination plant, under various synthesis temperatures and other conditions. This extracted Mg(OH)₂ is reported to have been used for synthesizing MgO at various temperatures and conditions at a relatively low cost.

Although MgO shows promising theoretical CO₂ adsorption rates, the capacities of reported CO₂ uptake of commercial bulk MgO, synthesized using various methods as well as by incorporating other materials with MgO, is still relatively low. The main obstacle in practical applications of MgO sorbent usage in CO₂ adsorption/mineralization lies in its lack of active sites which can be improved by achieving high surface area.

Thus, there is a need for effective methods to improve the performance of MgO for efficient CO₂ capture. Furthermore, other desirable features and characteristics will become apparent from the subsequent detailed description and the appended claims, taken in conjunction with the accompanying drawings and this background of the disclosure.

### SUMMARY

According to at least one aspect of the present embodiments, a method for fabrication of sorbents is provided. The method includes using calcination to obtain MgO—Mg(OH)₂ nano-composites and aging the MgO—Mg(OH)₂ nano-composites to form nano MCHs for room temperature carbon dioxide adsorption.

According to another aspect of the present embodiments, another method for fabrication of sorbents is provided. The method includes fabrication of monoclinic magnesium malate tetrahydrate (C₈H₁₀MgO₁₀.4H₂O) for room temperature carbon dioxide adsorption.

And according to a further aspect of the present embodiments, the use of monoclinic magnesium malate tetrahydrate (C₈H₁₀MgO₁₀.4H₂O) for room temperature carbon dioxide adsorption is provided.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with present embodiments.

FIG. 1 depicts an illustration of magnesium hydroxide powder produced in accordance with present embodiments.

FIG. 2 depicts a block diagram of a TGA analysis procedure for the MgO electrospun samples and MgO direct calcined samples produced in accordance with the present embodiments.

FIG. 3 depict a graph of variations in CO₂ adsorption over time at 30° C. for 1.5 hours of five samples in accordance with the present embodiments.

FIG. 4 depicts a graph of variations in CO₂ adsorption over time at 30° C. for various aging times of three samples in accordance with the present embodiments.

FIG. 5 depicts a graph of thermal decomposition of a six-month aged magnesium carbonate hydrate electrospun sample by TGA analysis in accordance with the present embodiments.

FIG. 6 depicts an illustration of a MgO—H₂O—CO₂ ternary phase diagram in accordance with the present embodiments.

FIG. 7A and FIG. 7B, depicts a X-ray diffraction (XRD) spectra for powders and electrospun films calcined at 300° C. in accordance with the present embodiments, wherein FIG. 7A depicts the XRD spectra for the powders and electrospun films and FIG. 7B depicts a magnification of the XRD spectra of FIG. 7A of 2θ in the range of 35 to 45 degrees.

FIG. 8 depicts a XRD spectra of three-month aged samples produced in accordance with the present embodiments.

FIG. 9A to FIG. 9C, depicts XRD spectra of the three-month aged electrospun samples indexed by C₈ (ICDD 00-052-2087) produced in accordance with the present embodiments, wherein FIG. 9A is a magnification of the XRD spectrum at 2θ in the range form 10° to 30°, FIG. 9B is a magnification of the XRD spectrum at 2θ in the range form 30° to 50°, and FIG. 9C is magnification of the XRD spectrum at 2θ in the range form 50° to 70°.

FIG. 10 depicts XRD spectra of the aged samples indexed by C₈H₁₀MgO₁₀.4H₂O in accordance with the present embodiments.

FIG. 11A to FIG. 11D, depicts energy dispersive spectroscopy (EDS) images for a six-month aged sample in accordance with the present embodiments, wherein FIG. 11A depicts an EDS image for the sample, FIG. 11B depicts an elemental mapping of carbon in the sample, FIG. 11C depicts an elemental mapping of oxygen in the sample and FIG. 11D depicts an elemental mapping of magnesium in the sample.

FIG. 12A and FIG. 12B, depicts SEM images of an electrospun sample calcined at 300° C. in accordance with the present embodiments, wherein FIG. 12A depicts an SEM images 11,000× magnification and FIG. 12B depicts an SEM images at 45,000× magnification.

FIG. 13A and FIG. 13B, depicts SEM images of a direct calcination sample produced at 300° C. in accordance with the present embodiments, wherein FIG. 13A depicts an SEM images 11,000× magnification and FIG. 13B depicts an SEM images at 45,000× magnification.

FIG. 14A and FIG. 14B, depicts SEM images of an electrospun sample calcined at 300° C. after the six-month aging treatment in accordance with the present embodiments, wherein FIG. 14A depicts an SEM images 11,000× magnification and FIG. 14B depicts an SEM images at 45,000× magnification.

Skilled artisans will appreciate that elements in the figures are illustrated for simplicity and clarity and have not necessarily been depicted to scale.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of present embodiments to present methods for fabrication of carbon dioxide capture magnesium oxide sorbents and magnesium oxide sorbents fabricated thereby for room temperature carbon dioxide adsorption. More particularly, it is the intent of the present embodiments to present use of monoclinic magnesium malate tetrahydrate synthesized using a novel method of combining electrospinning and aging processes to synthesize materials room temperature CO₂ adsorption and mineralization.

Effective solutions for efficient carbon dioxide (CO₂) capture at room temperature conditions are in high demand due to the major impact CO₂ has on global climate change. Among solid adsorbent materials for CO₂ capture, magnesium-based sorbents have been identified as promising sorbents for CO₂ capture at intermediate temperatures. In accordance with the present embodiments, a novel CO₂ mineralization approach is presented which leads to effective and practical solutions of carbon dioxide (CO₂) emission management and a great potential for novel carbon-based fuel development.

Using magnesium oxide (MgO) for CO₂ capture is not a new concept. Yet most conventional uses of MgO for CO₂ capture reported capture at high temperature ranges. For example, one previous method synthesized MgO nano/microparticles with multiple morphologies and porous structures via surfactant assisted solvothermal or hydrothermal route. The MgO derived with poly(ethylene glycol) showed around 3.68 wt % CO₂ uptake below 350° C., indicating high temperature CO₂ capture of MgO. Another previous synthesis prepared MgO with various porous structures at different calcination temperatures for CO₂ capture and compared them with commercial MgO. The sample calcined at 400° C. reported a 3.6 wt % capture capacity when compared to the 0.88 wt % reported for commercial MgO and suggested that the micropores of MgO can be optimized by controlling the calcination temperature for better adsorption rates in MgO. Another prior instance synthesized MgO by thermal decomposition and reported better adsorption rates at 50° C. with a high surface area.

However, there has been little discussion on CO₂ capture capacities of MgO at ambient temperature conditions. One study on the use of MgO with titanium oxide (TiO₂) for CO₂ adsorption at 25° C. via a sol-gel synthesis method showed better capture capacities than that of pure MgO or TiO₂. Also, morphology of MgO sorbents plays an important role in enhancing CO₂ capture capacities as evidenced by MgO synthesised by a prior aerogel method which reported around 10 wt % of CO₂ capture at 30° C., concluding that the sorption capacities are not directly proportional to the sorbent surface area. Additionally, incorporating alkali nitrates, nitrites, and carbonates with MgO is reported to improve CO₂ capture capacities at high temperature, allowing capture capacities of up to 19.8 wt %.

Carbon mineralization using magnesium extracted from brine has attracted much attention owning to its great potentials to provide a low cost, secured and permanent method to dispose CO₂. Besides MgO, numerous efforts have been made for CO₂ mineralization by using MCHs such as nesquehonite (MgCO₃.3H₂O), because they consist of at least 30% of carbonates (CO₃²⁻). A further study of formation of MgCO₃.3H₂O showed a CO₂ mineralization system synthesized at 20° C. by using Mg(OH)₂ as an intermediate which presented that a Mg(OH)₂ morphology has an effect on the growth rate, size and morphology of MgCO₃.3H₂O, thus a way to control crystallization of MgCO₃.3H₂O in CO₂ mineralization.

Apart from MgCO₃.3H₂O, the monoclinic magnesium malate tetrahydrate (C₈H₁₀MgO₁₀.4H₂O) or C₈ is even richer in CO₃²⁻. However, neither CO₂ adsorption nor CO₂ mineralization of C₈ has been investigated. Therefore, in accordance with the present embodiments, C₈ is utilized for CO₂ adsorption at room temperature. Commercially available Mg(OH)₂ is used as a precursor in electrospinning synthesis, and an aqueous solution of polyvinyl alcohol (PVA) is used as a polymer base to obtain nanofibers. The obtained nanofibers containing Mg(OH)₂ then underwent two types of synthesizing processes: (1) P1: using calcination to obtain MgO—Mg(OH)₂ nano-composites in a range of heating temperatures; (2) P2: using additional ageing treatment to form nano MCHs, wherein the additional aging treatment includes ageing the nanocomposites for a multi-month time such as three to six months. Characteristics of obtained samples were thoroughly investigated using thermogravimetric analysis (TGA), X-ray diffraction (XRD), scanning electron microscopy (SEM), and energy dispersive spectroscopy (EDS) analysis and phase diagram and thermodynamic analysis were used to propose mechanisms for thermal decomposition, CO₂ adsorption, and CO₂ mineralization.

For samples synthesized via the P1 process and the P2 process in accordance with the present embodiments, analytical grade PVA (Mw 89,000-98,000, 99+% hydrolyzed) and reagent grade Mg(OH)₂ 95% were purchased from Sigma-Aldrich (i.e., Millipore Sigma of the Merck Group of St. Louis, Mo., USA), and analytical grade glacial acetic acid (AA) 99.8% was purchased from Scharlau (i.e., Scharlab S.L. of Sentmenat, Spain). All the chemicals were used as received without further purification. Deionized water was used in all experiments.

An aqueous PVA (5% w/w) solution was prepared by dissolving PVA powder in distilled water, followed by stirring at 80° C. for two hours and then cooling to room temperature for another twelve hours under continuous stirring. Next, 0.25 g Mg(OH)₂ was dissolved in five ml acetic acid under sonication in a water bath at 55° C. for one hour. These two solutions were then mixed in 15:100 ratio (i.e., 0.750 ml aqueous PVA: 5 ml Mg(OH)₂-acetic acid) under further sonication in a water bath at 55° C. for twenty minutes to eliminate any precipitation.

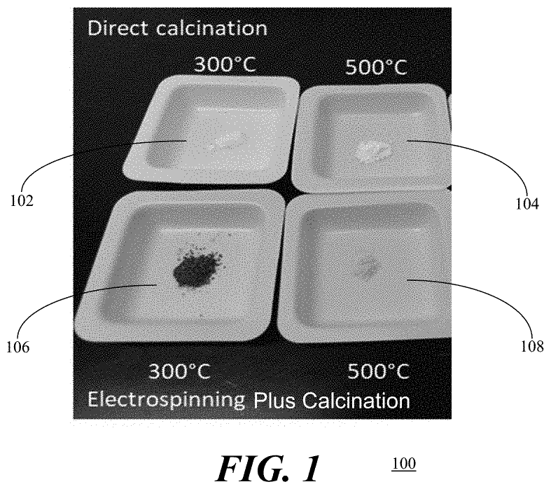

Electrospinning was carried out using a needle-collector setup in top-down configuration with aluminum foil spread across the collector plate. A twenty kilovolt (20 kV) voltage was applied over a 21G×½″ needle with a sharp end ground flat. The distance between needle and collector was kept at thirteen centimeters, and the flow rate was 0.3 ml/hrs. The nanofiber layer deposited on the aluminum foil was oven-dried at 60° C. for twenty-four hours to obtain a solidified layer with brittle consistency. The oven-dried nanofibers were then collected as flakes for calcination in a box furnace (an 1100 model furnace from Anhui Haibei Import & Export Co., Ltd., of Hefei City, Anhui province, PRC) at 300° C. and 500° C. During calcination, the furnace temperature was increased from 30° C. to 300° C./500° C. with a heating rate of 2° C. per minute. The samples were kept for two hours at 300° C./500° C. followed by air cooling to room temperature. As the calcination temperature increasing, the heat treatment produced coarse powder with lighter color, as pictured in FIG. 1. FIG. 1 depicts an illustration 100 of magnesium hydroxide powder produced in accordance with the present embodiments, wherein images 102, 104 depict magnesium hydroxide powder produced by direct calcination and images 106, 108 depict magnesium hydroxide powder produced by electrospinnng plus calcination.

The accompanying chemical reactions of the P1 synthesized samples are:

Mg(OH)₂(s)→MgO(s)+H₂O (1)

Mg(OH)₂(s)+CO₂(g)→MgCO₃(s)+H₂O(g) (2)

P2 samples were prepared by exposing the sample prepared by the P1 process to air at room temperature and ambient moisture conditions for three to six months. This aging treatment method was aimed to synthesize nuclear seeds of MCHs such that it may further fully grow into mineral crystals upon CO₂ up taking from air. Some of the hydrate candidates in the samples upon aging are listed as follows: nesquehonite MgCO₃.3H₂O(N), hydromagnesite 4MgCO₃.Mg(OH)₂.4H₂O (HY), dypingite 4MgCO₃.Mg(OH)₂.5H₂O, artinite MgCO₃.Mg(OH)₂.3H₂O, and lansfordite MgCO₃.5H₂O.

During the aging process, CO₂ from the air and the breakdown residuals of PVA may act as the principal carbon source for the nucleation or mineralization of some of the above-listed hydrated magnesium carbonates. Subsequently, these nucleation sites may provide additional avenues for the CO₂ capture and mineralization process, thus increasing the measured capacity significantly. This phenomenon may advantageously be applied to advance new research in CO₂ capture, CO₂ mineralization and new carbon-based fuels.

X-ray diffraction (XRD) measurements of test samples were conducted using a Bruker D8 Advance X-ray diffractometer (from Bruker Corporation of Billerica, Mass., USA) with Cu-Kα radiation of 1.54 Å to evaluate powder composition and phase. The scanning angle was adjusted between 2θ angles (from 10° to 70°) with the X-ray generator running at an applied voltage of forty kilovolts (40 kV) and a current of 25 milliamps (25 mA). Surface structure and morphology were examined by scanning electron microscopy (SEM) (JEOL JSM-7600F from JEOL Ltd of Tokyo, Japan), and the elemental analysis imaging was done by using energy-dispersive spectroscopy (EDS), (Oxford Instruments X-MaxN-50 from Oxford Instruments of Abingdon-on-Thames, England) embedded in SEM.

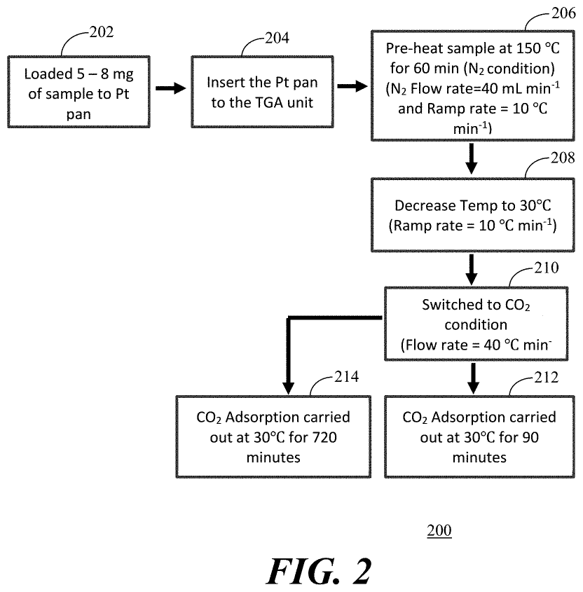

Thermogravimetric analysis (TGA) of the samples for CO₂ capture and thermal decomposition of the aged samples was conducted using a TGA Q50 analyser (from TA Instruments, a wholly owned subsidiary of Waters Corporation of Milford, Mass., USA). By TGA tests, the CO₂ capture capacity of the samples from the P1 and P2 processes was measured at, respectively, 3.9% and 14.7% at room temperature. Referring to FIG. 2, a block diagram 200 depicts a TGA analysis procedure for the MgO electrospun samples and MgO direct calcined samples produced in accordance with the present embodiments. TGA analysis was begun by loading 202 five to eight milligrams to a platinum (Pt) pan which is inserted 204 into the TGA unit for CO₂ adsorption performance measurement. To avoid errors caused by pre-adsorbed species such as atmospheric CO₂, water and other impurities, samples were pre-treated 206 at 150° C. for sixty minutes under a flow of high purity N₂ (40 mL min⁻¹) with a ramp rate of 10° C. per minute. The temperature was then lowered 208 to the desired adsorption temperature (e.g., 30° C.) at a rate of 10° C. per minute, the gas was switched 210 from N₂ to CO₂ with a constant flow of pure CO₂ (one atmosphere, 40 mL and the CO₂ adsorption uptake was measured for ninety minutes initially 212 and then measured for 720 minutes 214.

A thermal decomposition analysis was carried out by heating a six-month aged sample from 30° C. to 500° C. at a continuous flow of compressed dry air. The flow rate of the dry air was kept at forty millilitres per minute (40 mL min′) with a ramp rate of 10° C. per minute. The sample was held at 500° C. and a constant compressed dry air with a flow rate of 40 mL min⁻¹ for one hour to obtain a time-dependent weight loss profile and phase transitions during thermal decomposition.

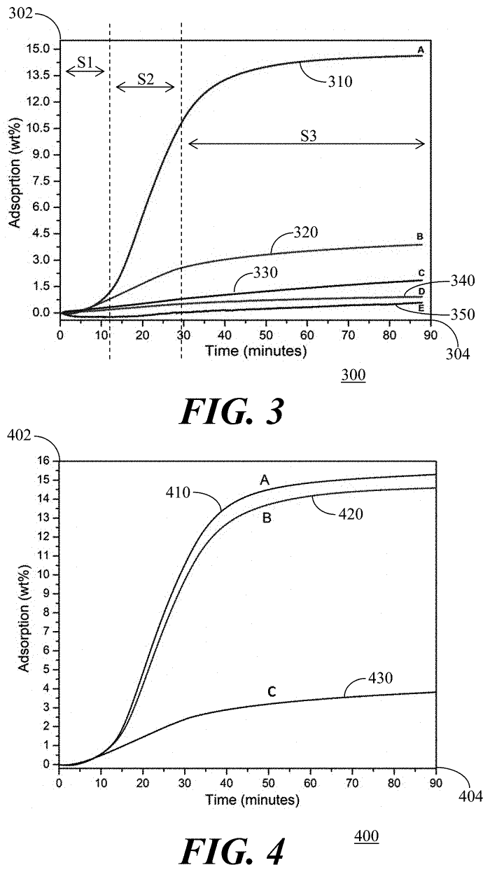

For each sample, CO₂ adsorption capacity was measured using the Q50 TGA analyser. The sample with a weight in the range of five to eight milligrams was analysed at 30° C. with a constant flow of high purity CO₂ for 1.5 hours as longer time periods may not reveal any important information for practical applications. Referring to FIG. 3, a graph 300 depicts variations in CO₂ adsorption 302 over time 304. A curve 310 refers to a three-month aged electrospun sample of magnesium carbonate hydrates (MCH) calcined at 300° C. A curve 320 refers to an electrospun sample calcined at 300° C. A curve 330 refers to a sample produced by direct calcination (not electrospun) at 500° C. A curve 340 refers to a sample produced by direct calcination at 300° C. And a curve 350 refers to an electrospun sample calcined at 500° C.

The results indicate that the electrospun samples calcined at 300° C. and 500° C. (the curves 320 and 350, respectively) achieved adsorptions up to 3.9 wt % and 0.5 wt %, respectively, within ninety minutes. However, the samples obtained by direct calcination at 300° C. and 500° C. (the curves 330 and 340, respectively) delivered CO₂ adsorptions up to only 0.9 wt % and 1.9 wt %, respectively, within ninety minutes. Differences in the samples' chemistry (i.e., MgO:Mg(OH)₂ ratio) and microstructure may be the source of the observed inverse-temperature-dependence in CO₂ absorption. The CO₂ capacity captured by the three-month aged electrospun sample of MCH calcined at 300° C. (the curve 310) produced in accordance with the present embodiments was advantageously up to 14.6 wt % within ninety minutes. CO₂ capture within ninety minutes of the samples produced in accordance with the represent embodiments was achieved through a three-stage dynamic process as shown in the curve 310: S1: a short incubation period in the first twelve minutes; S2: a rapid increase in absorption rate (approximately 0.5 wt % per minute) between twelve to thirty minutes, where adsorption reaches 10 wt %; and S3: a saturation period from thirty to ninety minutes. A sharp increase in CO₂ adsorption observed in the three-month aged sample (the curve 310) may suggest the formation of a new phase(s) or structure(s) which has not previously reported in MgO sorbent systems.

FIG. 4 depicts a graph 400 of variations in CO₂ adsorption 402 over time 404 at 30° C. for various aging times of three samples in accordance with the present embodiments. A curve 410 refers to a six-month aged magnesium carbonate hydrate electrospun sample. A curve 420 refers to a three-month aged magnesium carbonate hydrate electrospun sample. And a curve 430 refers to the same magnesium carbonate hydrate electrospun sample prior to aging treatments.

For the six-month aged MCH electrospun sample (the curve 410), the values of CO₂ adsorption capacity in the forty-minute and ninety-minute range were 14 wt % and 15.5 wt %, respectively, through the aforementioned three-stage dynamic process. Interestingly, the three-month prolonged aging period (the curve 420) resulted in an additional ˜1 wt % increase in CO₂ absorption and approximately eight minute decrease in absorption time. Thus, some common crystal phase(s) or structure(s) may be expected to dominate the CO₂ adsorption/mineralization behaviours in both the three-month and six-month aged MCH electrospun samples. Therefore, further XRD measurements of test samples were conducted to evaluate their crystal structures.

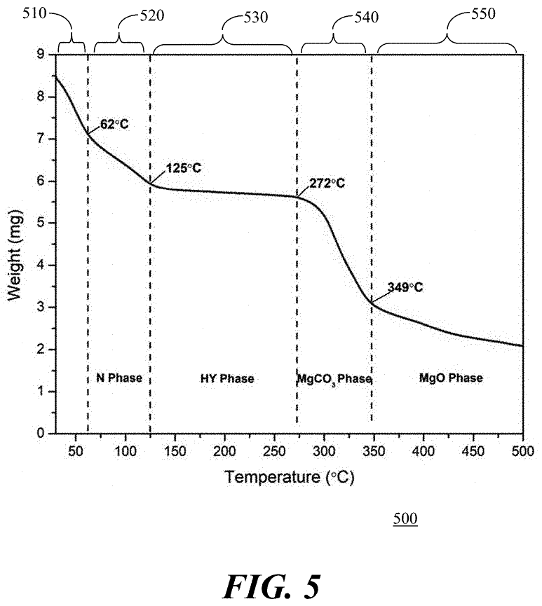

Referring to FIG. 5, a graph 500 presents the thermal decomposition of the six-month aged MCH electrospun sample, based on TGA analysis, to reveal its thermal decomposition during the aging process at room temperature. In the graph 500, five distinctly different zones can be identified as follows: Z₁ (from 25° C. to 62° C.) 510, Z₂ (from 62° C. to 125° C.) 520, Z₃ (from 125° C. to 272° C.) 530, Z₄ (from 272° C. to 349° C.) 540, and Z₅ (from 349° C. to 500° C.) 550.

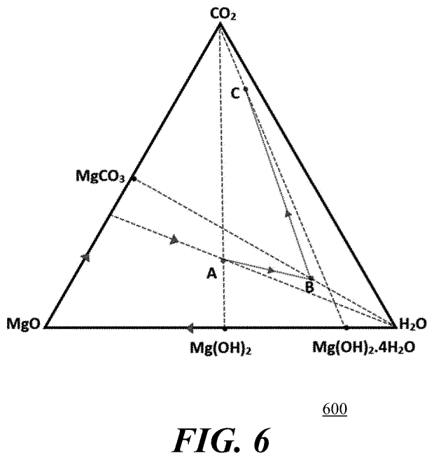

The thermal stability of test sample in Zᵢ (i=1 to 5) 510 was associated with the observed temperature-dependent weight-loss or the slope of the curve within that zone. Therefore, a rank of a sample's thermal stability (high to low) in the temperature range from 25° C. to 500° C. was obtained: Z₃>Z₅>Z₂>Z₁=Z₄. There was a slight slope change within Z₁ (at −37° C.) and Z₄ (at −300° C.). Apart from that, a highly stable plateau in Z₃ was observed. These observations were consistent with phase diagram analysis shown in FIG. 6, thermodynamic calculations, and literature reports.

All materials in this study can be represented by using a MgO—H₂O—CO₂ ternary phase diagram 600 as depicted in FIG. 6. The phase diagram 600 presents the phase relationship in the studied materials systems in which the symbol A represents the ternary compound 4MgCO₃.Mg(OH)₂.4H₂O, the symbol B represents the binary compound MgCO₃.3H₂O (in between MgCO₃ and H₂O), and the symbol C represents the projection of the quaternary compound C₈. The [C₄H₈] group is not shown on the ternary plane but a sub C—H binary (not shown here), whose C/H ratio is 0.5.

According to previous reports magnesite (MgCO₃) is decomposed into MgO and CO₂ at −350° C., which is very close to the TGA experimental results presented hereinabove and a calculated value of 349° C. using FactSage™, a thermochemical database system from GTT-Technologies of Aachen, Germany. This transition between Z₅ and Z₄ in the graph 500 represents the MgO rich side of the MgO—CO₂ sub-binary diagram in the phase diagram 600.

The transition temperature at 272° C. between Z₄ and Z₃ is in agreement with previous reports that 4MgCO₃.Mg(OH)₂.4H₂O dehydration took place in the range of 200° C. to 300° C. under a high carbon dioxide pressure (e.g., 21 kg cm⁻²). Interestingly, there is a smooth slope change at −300° C., as shown in the zone Z₄ 540 in the graph 500. This can be explained by a weak surface 4MgCO₃.Mg(OH)₂.4H₂O dehydration between 272° C. and 300° C., followed by active bulk HY dehydration between 300° C. and 349° C. 4MgCO₃.Mg(OH)₂.4H₂O is a ternary compound shown in point A in the phase diagram 600, and its formation and decomposition rate strongly depends on the diffusion coupling among CO₃²⁻, Mg²⁺, and OH⁻ ions to maintain the stoichiometry of 4MgCO₃.Mg(OH)₂.4H₂O. This phenomenon can be explained by using excellent thermos-stability in the long plateau of Z₃ 530, as shown in the graph 500.

Furthermore, a phase transition has been reported of MgCO₃.3H₂O to a stable amorphous magnesium carbonate at about 115° C., which was ten degrees lower than the transition temperature (125° C.) between Z₂ 520 and Z₃ 530 in the graph 500. Since this amorphous phase had a chemical composition close to that of 4MgCO₃.Mg(OH)₂.4H₂O, it was expected that this resultant ten-degree difference reflected the structural variety of amorphous phases associated with individually applied synthesis parameters. Correspondingly, the phase transition between 4MgCO₃.Mg(OH)₂.4H₂O and MgCO₃.3H₂O was shown from point A to B in the phase diagram 600. The transition temperature between Z₁ 510 and Z₂ 520 at 62° C. was also in agreement with previous reports, in which MgCO₃.3H₂O was precipitated below 52° C. Since, the ten-degree difference in temperature is outside the normal experimental error limits, it indicates that Z₁ 510 is not for a pure single-phase zone but for a composite with minor N phase addition. In addition, XRD characterization was conducted to index the structures of the three-month and six-month aged samples, as discussed hereinafter.

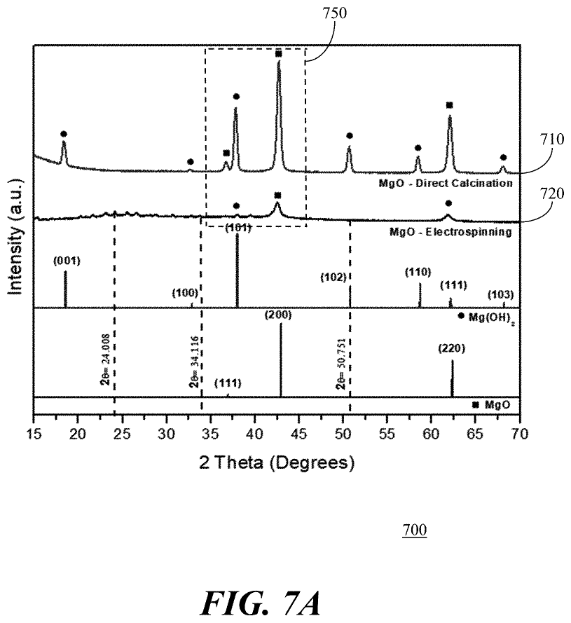

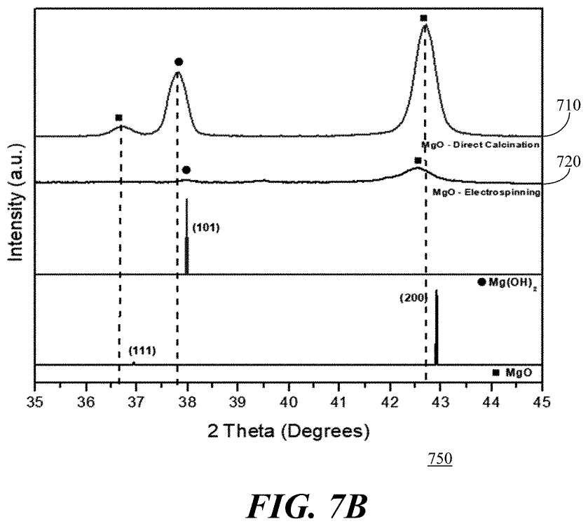

FIG. 7A presents XRD spectra for powders and electrospun films calcined at 300° C., and symbols solid square and circle respectively represent MgO and Mg(OH)₂. The major peak positions of MgO (111), MgO (200), MgO (220), Mg(OH)₂ (001), Mg(OH)₂ (100), Mg(OH)₂ (101), Mg(OH)₂ (102), Mg(OH)₂ (110), and Mg(OH)₂ (103) indexed the formation of MgO (ICDD 00-045-0946) as well the residual Mg(OH)₂ (ICDD 00-044-1482) in the MgO direct calcination samples. Similarly, the MgO (200), Mg(OH)₂ (101), and Mg(OH)₂ (111) peaks with much lower intensities and larger widths demonstrated the presence of both MgO and Mg(OH)₂ in the electrospun samples.

Referring to FIG. 7A, a XRD spectra 700 is depicted for powders 710 and electrospun films 720 calcined at 300° C. in accordance with the present embodiments. FIG. 7B depicts a magnification 750 of the XRD spectra 700. The magnification 750 depicts a peak shift from 36.947° to 36.674° for (111) peak, 37.984° to 37.819° for (101) peak, 42.909° to 42.709° for (200) peak in the powder 710 calcinated at 300° C., and shift from 42.909° to 42.525° for (200) peak and shift from 37.984° to 38.085° for (101) peak in the electrospun films 720 calcinated at 300° C.

Numerous small peaks (eg: 2θ=24°) were also detected in the XRD analysis of electrospun samples, yet they were absent in the direct calcination samples. This indicated that the sample's residual polymer fragments possibly contained carbon element originating from the PVA of the electrospinning solutions. These residuals may serve as nucleation centres for CO₂ adsorption/mineralization. However, their lower intensities compared to those of MgO and Mg(OH)₂, ruled out the possibility that these residuals were the carbon source needed for the formation of new mineral phases during aging. Nevertheless, low intensity and large width peaks of electrospun MgO sample indicates the poor crystallinity, which may suggest a defect rich and amorphous like-MgO structure

For direct calcination samples 710 at 300° C., shifts in XRD peak position were detected as follows: from 37.984° to 37.819° for Mg(OH)₂ (101) (ICDD 00-044-1482), and from 42.909° to 42.709° for MgO (200) (ICDD 00-045-0946), as shown in the XRD spectra 750. Similar peak shifting also was observed in the electrospun MgO samples 720: from 42.909° to 42.525° for MgO (200) and 37.984° to 38.085° for Mg(OH)₂ (101). It has been discussed that these peak shifts may be caused by subtle differences in x-ray transparency of the specimens due to the air scattering of photons. The larger reduction of 2θ angles for MgO (200) is consistent with the effect of nano-surface relaxation in the electrospun samples 720.

In summary, MgO/Mg(OH)₂ composites were detected in both electrospun samples 720 and direct calcined samples 710, and the trace of residual polymer fragments from the precursor solution was expected in the electrospun samples 720. However, the traces of polymer residuals were ruled out to serve as the carbon sources for CO₂ mineralizing during the aging process as only other carbon element source available during the mineralization process is CO₂ in air.

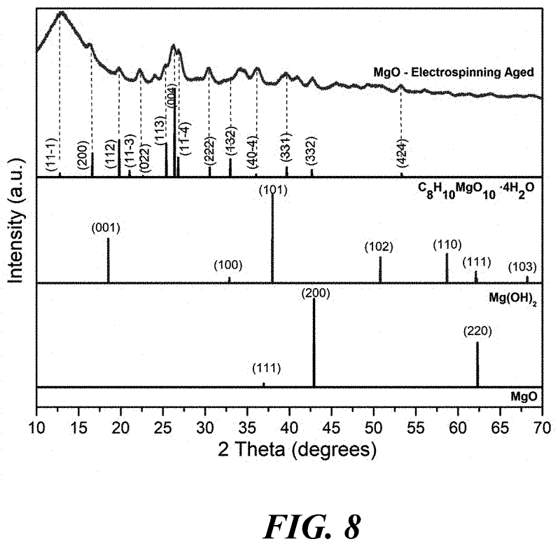

XRD measurements were also carried out to study the mineralization process of the three-month aged electrospun samples. FIG. 8 depicts a XRD spectra 800 for the three-month aged electrospun samples produced in accordance with the present embodiments. In the XRD spectra 800, peaks at 2θ=12.79°, 19.81% 26.29°, 26.81% 30.48°, 32.99°, 36.08°, 39.68°, and 53.30° are respectively consistent with (111̅), (112), (004), (114̅), (222), (132), (404̅), (331), and (424) of CsMg (HO)₁₀.4H₂O (ICDD 00-052-2087). The XRD spectra 800 also shows that the Mg(OH)₂ phase was dissolved in the aged samples, indicated by the disappearance of its (001), (100), (101), (102), (110), (111) and (103) diffraction peaks. However, the presence of MgO (111) and (200) peaks suggests the existence of a stable MgO phase.

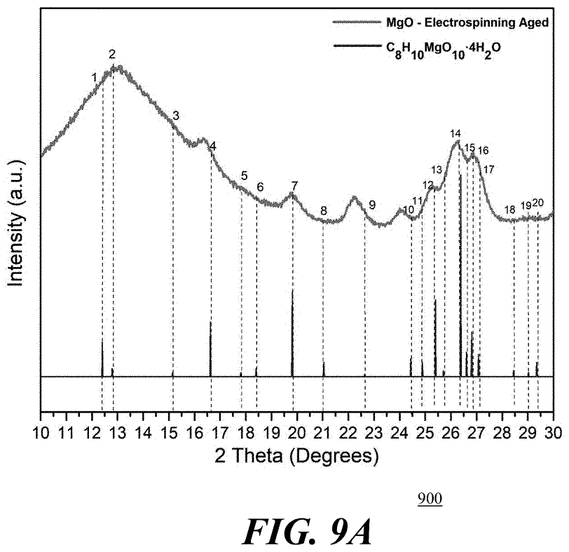

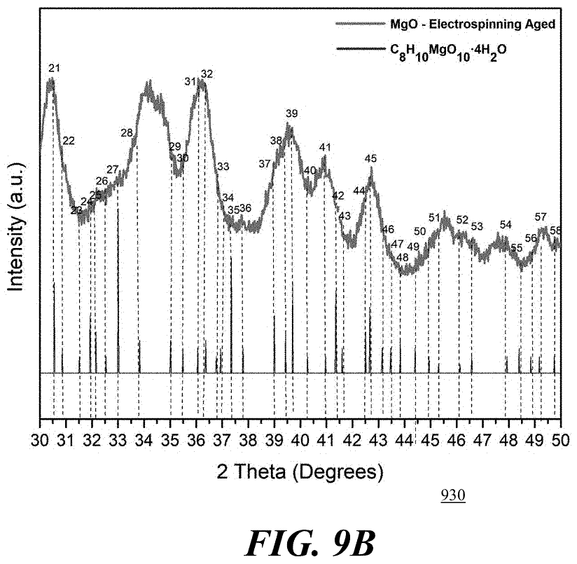

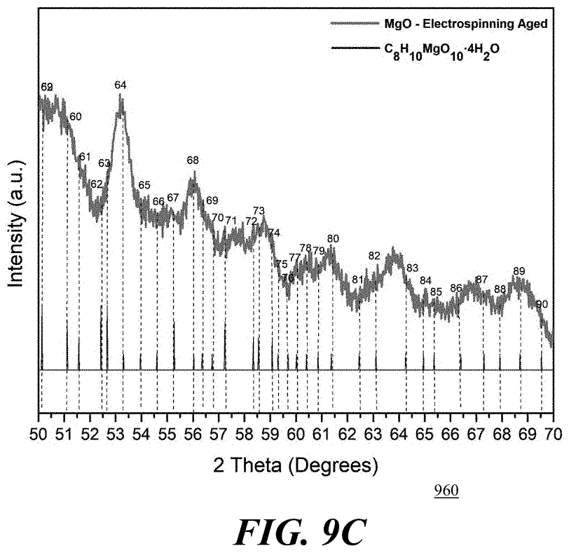

FIGS. 9A, 9B and 9C depict magnified XRD spectrum 900, 930, 960 of a three-month aged electrospun sample produced in accordance with the present embodiments and indexed by C₈ (ICDD 00-052-2087). The magnification 900 of the XRD spectrum is magnified at 2θ in the range from 10° to 30°. The magnification 930 of the XRD spectrum is magnified at 2θ in the range from 30° to 50°. And the magnification 960 of the XRD spectrum is magnified at 2θ in the range from 50° to 70°.

To characterize the newly formed phases in the aged samples, TABLE 1 lists five available XRD spectrum data of hydrates as follows: (1) Mg₂CO₃(OH)₂.3H₂O, (2) C₈Mg(HO)₁₀.4H₂O, (3) Mg₃(CO₃)₄(OH)₂.4H₂O, (4) Mg₄(OH)₂(CO₃)₃.3H₂O, and (5) MgCO₃.3H₂O. The XRD data of C₈ exhibited the best fit, compared with the data in the XRD spectra 800, 900, 930, 960, where only one peak located between 34° and 36° is not represented. The highest measured peak of this compound (the XRD spectra 800), at about 2θ=13° did not match the relative intensity of the reference data; however, the second highest peak at about 2θ=26.4° and its neighbouring peaks were well represented.

TABLE 1

| Name | Mg/H₂O | C/H₂O | OH/H₂O | First | Second | Third | Fourth |
|---|---|---|---|---|---|---|---|
| Mg₂CO₃(OH)₂•3H₂O | 0.667 | 0.333 | 0.667 | 32.71/100 | 16.59/65 | 24.10/50 | 40.80/40 |
| C₆H₆O₆C₂H₂O₂•Mg(OH)₂•4H₂O | 0.25 | 2 | 2.5 | 26.39/100 | 19.81/42 | 25.4/38 | 16.63/27 |
| Mg₃(CO₃)₄ (OH)₂•4H₂O | 0.75 | 1 | 0.5 | 15.29/100 | 30.78/90 | 41.97/50 | 13.74/40 |
| Mg₄(OH)₂ (CO₃)₃•3H₂O | 1.333 | 1 | 0.667 | 15.29/100 | 30.81/90 | 41.99/50 | 13.74/40 |
| MgCO₃•3H₂O | 0.333 | 0.333 | 0 | 13.65/100 | 23.08/75 | 34.24/55 | 29.46/30 |

The XRD data for the five selected hydrates in TABLE 1 indicates the chemical ratio and the 2θ over intensity for the four highest peaks (e.g., 32.71/100 refers to the 2θ over intensity where at 2θ=32.71 the peak intensity is 100%). Further analysis of the five compounds listed in Table 1 indicated a strong presence of MgCO₃.3H₂O since its highest peak at 2θ=13.65 and third highest peak at 2θ=34.24° do not belong to C₈ to match those of the three-month aged samples as shown in the XRD spectra 800. In addition, as a result of the magnification of the XRD spectrum 900, 930, 960, it is hypothesized that the three-month aged sample was a nanocomposite comprised of C₈, MgCO₃.3H₂O, and residual MgO.

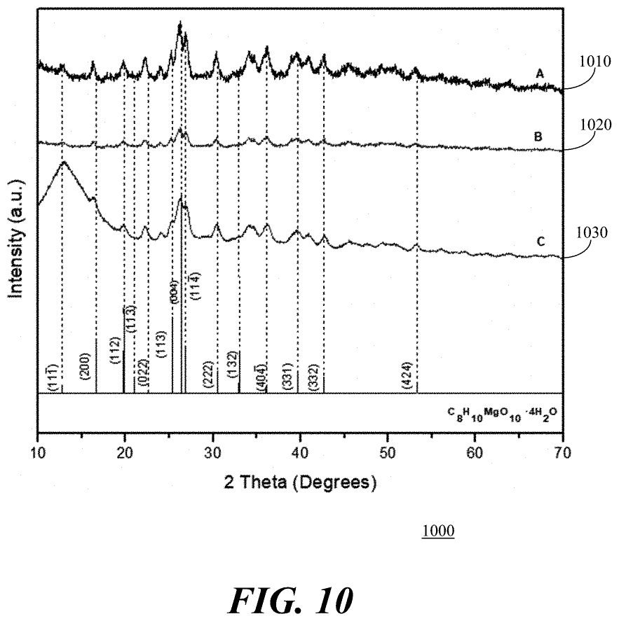

Referring to FIG. 10, XRD spectra 1000 of aged samples indexed by C₈ are depicted. A curve 1010 is a XRD spectrum of a six-month aged sample after TGA-CO₂ analysis. A curve 1020 is a XRD spectrum of a six-month aged sample before TGA-CO₂ analysis. And a curve 1030 is a XRD spectrum of a three-month aged sample. The curve 1030 indicates two phases (N and C₈) in the reference three-month aged sample. The curve 1010 and the curve 1020 present the C₈ single phase in six-month aged samples after and prior to the TGA-CO₂ analysis, respectively. After the TGA-CO₂ test, no significant structural changes were observed in the six-month aged samples, although higher and broader peaks in the curve 1010 may suggest enhanced carbon diffusion into the lattices. The most striking observation in XRD spectra 1000 was the almost full transformation of the MgCO₃.3H₂O appearing in the three-month aged sample (the curve 1030) into C₈ in the six-month aged ones the curves 1010 and 1020). This trend likely follows the route from point B to point C in the phase diagram 600. Therefore, the zone Z₁ 510 in the graph 500 (FIG. 5) was dominated by C₈.

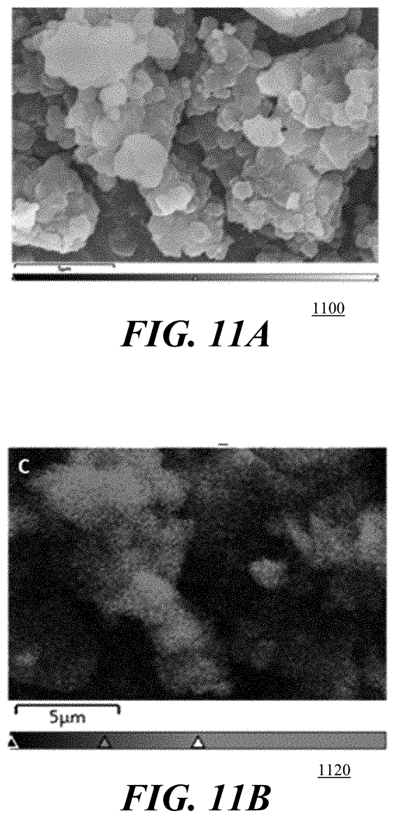

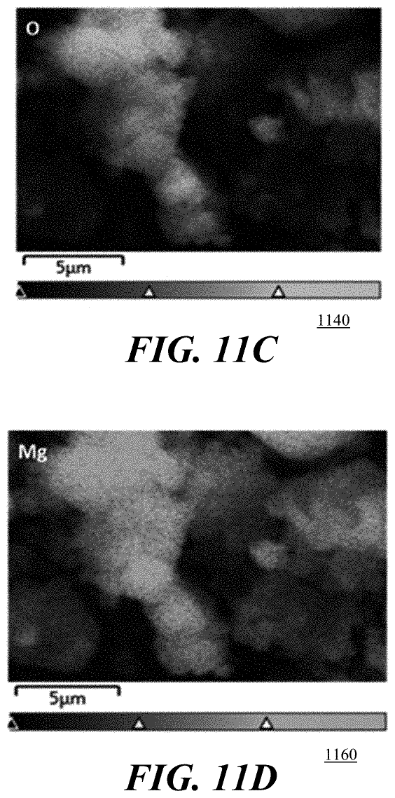

Referring to FIGS. 11A, 11B, 11C and 11D, energy dispersive spectroscopy (EDS) images 1100, 1120, 1140, 1160 for the six-month aged sample indicate the presence of a single-phase containing at least Mg, O, and C elements. The EDS image 1100 depicts energy dispersive spectroscopy of the sample, the EDS image 1120 depicts elemental mapping of carbon in the sample, the EDS image 1140 depicts elemental mapping of oxygen in the sample, and the EDS image 1160 depicts elemental mapping of magnesium in the sample. The elemental distribution of magnesium in the EDS image 1160 well matched the elemental distribution of oxygen in the EDS image 1140 and the elemental distribution of carbon in the EDS image 1120, suggesting a single-phase structure.

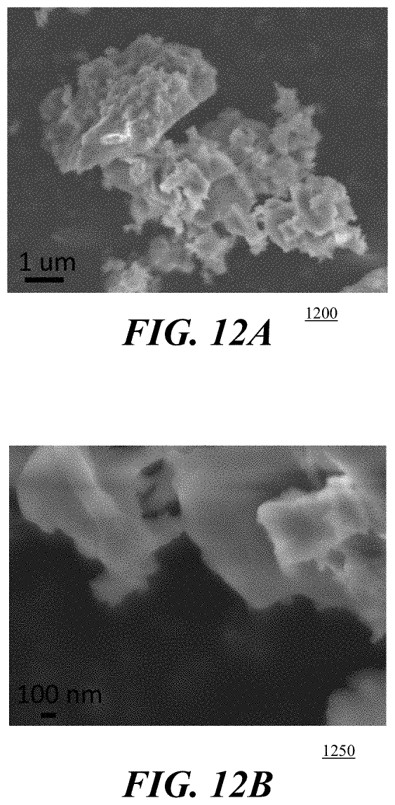

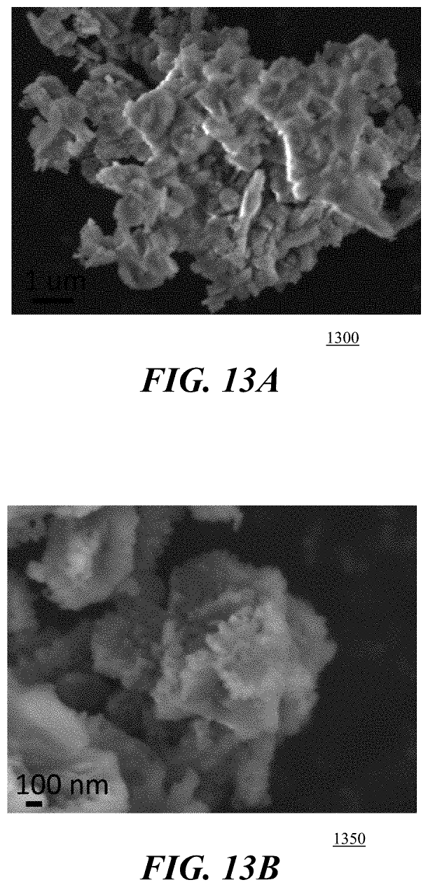

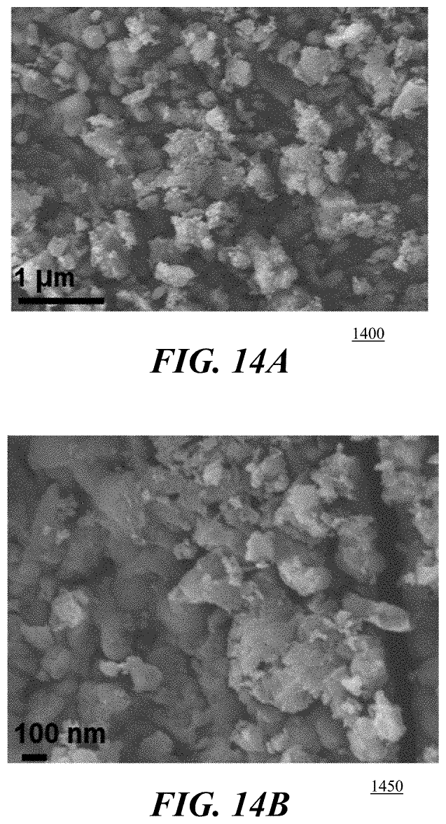

Referring to FIGS. 12A and 12B, SEM images 1200, 1250 depict SEM images of an electrospun sample calcined at 300° C. in accordance with the present embodiments, wherein the SEM image 1200 is depicted at 11,000× magnification and the SEM image 1250 is depicted at 45,000× magnification. Referring to FIGS. 13A and 13B, SEM images 1300, 1350 depict SEM images of a direct calcination sample produced at 300° C. in accordance with the present embodiments, wherein the SEM image 1300 is depicted at 11,000× magnification and the SEM image 1350 is depicted at 45,000× magnification. Referring to FIGS. 14A and 14B, SEM images 1400, 1450 depict SEM images of an electrospun sample calcined at 300° C. after the six-month ageing treatment in accordance with the present embodiments, wherein the SEM image 1400 is depicted at 11,000× magnification and the SEM image 1450 is depicted at 45,000× magnification.

As shown in the SEM images 1200, 1250 and the SEM images 1300, 1350, electrospun samples and direct calcined samples exhibited dissimilarities in morphology from the aged samples in the images 1400, 1450. The electrospun sample (SEM images 1200, 1250) appeared to have hierarchical sheet-like structures with a relatively smooth surface. In contrast, the direct calcination sample (SEM images 1300, 1350) was observed to be more granular, with features varying from 1 nm to 20 nm. The morphology of the sample in the SEM images 1400, 1450 that underwent aging treatment shows sheet-like structures with smooth surfaces.

Based on the above TGA, XRD, SEM, EDS analysis, phase diagram and thermodynamic calculations, as well as literature reports, three mechanisms were proposed as follows:

Mechanism of Thermal Decomposition of Six-Month Aged Electrospun Products (M1)

Based on thermal decomposition of the six-month aged MCH sample by TGA analysis (the graph 500, FIG. 5), the mechanism of thermal decomposition (M1) is described as follows:

M1-1: From 25° C. to 37° C., the surface desorption of gaseous molecules (CO₂, H₂O, and C₄H₈) from C₈ into air was shown in Z₁ 510 and point C in the phase diagram 600 (FIG. 6).

M1-2: From 37° C. to 62° C., the lattice desorption of gaseous molecules from C₈ into air was shown in Z₁ 510 and point C in the phase diagram 600.

M1-3: From 62° C. to 125° C., the formation of MgCO₃.3H₂O (from Z₁ 510 to Z₂ 520 and from point C to B in the phase diagram 600) was achieved by releasing one H₂O, four CO₂ and one C₄H₈ per C₈ molecule.

M1-4: From 125° C. to 272° C., the formation of 4MgCO₃.Mg(OH)₂.4H₂O amorphous (from Z₃ 530 to Z₄ 540 and from point B to A in the phase diagram 600) was achieved by structural reactions among MgO, H₂O, and CO₂ molecules.

M1-5: From 272° C. to 300° C., the formation of surface MgCO₃ was achieved by surface structural modifications, as shown in Z₄ 540 and point A in the phase diagram 600, driven by nano-surface relaxation effect of the electrospun samples.

M1-6: From 300° C. to 349° C., the formation of lattice MgCO₃ was achieved via dehydration delayed by the additional inward lattice diffusion process.

M1-7: From 349° C. to 500° C., the formation of MgO was achieved, by further releasing CO₂ molecules, as shown in Z₅ 550 and near MgO end on the MgO—CO₂ binary in the phase diagram 600.

Mechanism of CO₂ Adsorption of Six-Month Aged Electrospun Products (M2)

Based on CO₂ adsorption of the six-month aged MCH electrospun sample, the mechanism of CO₂ adsorption (M2) is described as follows:

M2-1: At 25° C., 3-step CO₂ adsorption was carried out: S1 (surface CO₂ adsorption), S2 (lattice CO₂ diffusion), and S3 (saturation). The 3-step adsorption is expected thermally reversible as in Z₁ 510. The obtained exceptionally high CO₂ capture capacity from the aged electrospun samples is believed to be due to the high CO₃²⁻ lattice occupancy in C₈.

Mechanism of CO₂ Mineralization of Six-Month Aged Electrospun Products (M3)

The CO₂ mineralization may be regarded as an inverse process of the thermal decompositions, and its mechanism (M3) is described as follows:

M3-1: Beginning from Mg(OH)₂ as the raw material, MgO was formed along the MgO—H₂O binary boundary in the phase diagram 600 under a calcination process at 300° C. Then, a nanocomposite of MgO and Mg(OH)₂ was detected, as shown in the XRD spectra 700, 750.

M3-2: At an early stage of the three-month aging process, by being exposed to H₂O and CO₂ molecules in air at room temperature, the electrospun MgO reacted with CO₂ along the MgO—CO₂ binary line of in the phase diagram 600, and the obtained product was a two-phase mixture of MgCO₃ and MgO.

M3-3: Further in the aging process, more CO₂, and H₂O molecules reacted with the MgCO₃ and MgO two phases resulting in the formation of the 4MgCO₃.Mg(OH)₂.4H₂O phase, which is located at point A within the triangle of MgO—MgCO₃—H₂O in the phase diagram 600.

M3-4: By a prolonged aging, the MgCO₃.3H₂O phase was formed as observed in the XRD spectra 700, 750, which followed the reaction route from point A to B in in the phase diagram 600. This formation was also aided by H₂O adsorption and CO₂ release.

M3-5: Finally, C₈ was formed by consuming MgCO₃.3H₂O phase with the addition of CO₂ molecules from the air and possibly their resultant C—H compounds like C₄H₈, as shown in the XRD spectra 1000. The MgCO₃.3H₂O was almost decomposed after a six-month aging treatment, which was also demonstrated from point B to C in the phase diagram 600.

Due to low CO₂ concentrations in air, the above mineralization mechanism progressed slowly in samples prepared by the P1 process. By spanning a duration from three-months to six-months in this study (i.e., the P2 process), much faster kinetics enabled the formation of nanostructures. This nanostructure-aided natural aging process can be sped up in a controlled environment. A pre-heating process in TGA tests, to prevent potential effects from moisture, was used at 150° C. for sixty minutes under N₂ condition. In the XRD spectra 900, 930, 960, an XRD peak indicating the presence of MgO was observed. The waste of magnesium sources in terms of CO₂ capture was produced due to its slow kinetics. For an optimal design, the aging process needs to be controlled to convert all MgO nanoparticles into the C₈ phase as soon as possible.

Finally, the energy consumption for regeneration of the sorbent based on chemical thermodynamic calculations was investigated using FactSage™. Since thermodynamic data for C₈ is not available, MgCO₃ was used to demonstrate the thermal regeneration of the proposed magnesium carbonate hydrate sorbent. One mole of pure MgCO₃ decomposes to one mole MgO and one mole CO₂ at 378° C. However, at 349° C. (i.e., 29° C. lower than 378° C.), one mole of chemicals with the same composition as compound C₈ produces one mole of MgO and four moles of a gas phase. Most importantly, a 29° C. temperature drop in the regeneration processing results in reducing the energy consumption for the regeneration of the sorbent. Furthermore, the released gas phase is a mixture of H₂O and CO₂, which can be further separated if desired.

A two-stage synthesized material that absorbed and mineralized CO₂ was designed, characterized, and validated. For the first time, the synthesis of MgCO₃.3H₂O, and C₈ was achieved by using a novel synthesis route that combined electrospinning and aging processes. Results revealed an impressive CO₂ capture capacity of 15.5 wt % using the proposed two-stage synthesized material at 30° C. Furthermore, the mechanisms for CO₂ adsorption/mineralization and thermal decomposition were postulated, based on TGA, XRD, SEM, and EDS analysis data in conjunction with thermodynamic calculations and MgO—CO₂—H₂O ternary phase diagram studies. The most striking observation was that, in a six-month aged sample, MgCO₃.3H₂O which appeared in the three-month aged sample was almost fully transformed into C₈. Materials produced in accordance with the present embodiments can provide advantageously improved carbon capture which can lead to CO₂ mineralization and to new carbon-based fuels.

Thus, it can be seen that the present embodiments provide effective methods to improve the performance of MgO for efficient CO₂ capture. Effects of calcination temperature and duration of aging on room temperature CO₂ adsorption, crystallization, and mineralization on sorbents showed that six-month aged electrospun samples of calcined MCH recorded a CO₂ adsorption capacity of 15.5 wt % within ninety minutes at 30° C.

While exemplary embodiments have been presented in the foregoing detailed description of the present embodiments, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing exemplary embodiments of the invention, it being understood that various changes may be made in the function and arrangement of steps and method of operation described in the exemplary embodiments without departing from the scope of the invention as set forth in the appended claims.

What is claimed is:

1. A method for fabrication of sorbents comprising:
using calcination to obtain MgO—Mg(OH)₂ nano-composites; and
aging the MgO—Mg(OH)₂ nano-composites to form nano MCHs for room temperature carbon dioxide adsorption.

2. The method in accordance with claim 1 further comprising before calcination using Mg(OH)₂ as a precursor in electrospinning synthesis.

3. The method in accordance with claim 1 wherein using calcination to obtain MgO—Mg(OH)₂ nano-composites comprises using an aqueous solution of polyvinyl alcohol (PVA) as a polymer base to obtain the MgO—Mg(OH)₂ nano-composites.

4. The method in accordance with claim 1 wherein the nano MCHs comprise nanofibers.

5. The method in accordance with claim 1 wherein using calcination to obtain MgO—Mg(OH)₂ nano-composites comprises calcination at a temperature of 300° C.

6. The method in accordance with claim 1 wherein using calcination to obtain MgO—Mg(OH)₂ nano-composites comprises calcination at a temperature of 500° C.

7. The method in accordance with claim 1 wherein aging the MgO—Mg(OH)₂ nano-composites comprises ageing the MgO—Mg(OH)₂ nano-composites for a multi-month time duration.

8. The method in accordance with claim 7 wherein the multi-month time duration comprises six months.

9. The method in accordance with claim 7 wherein the multi-month time duration comprises a time duration between three months and six months.

10. A method for fabrication of sorbents comprising fabrication of monoclinic magnesium malate tetrahydrate (C₈H₁₀MgO₁₀.4H₂O) for room temperature carbon dioxide adsorption.

11. The method in accordance with claim 10 wherein the C₈H₁₀MgO₁₀.4H₂O comprises nanofibers.

12. The method in accordance with claim 10 wherein the C₈H₁₀MgO₁₀.4H₂O is formed by a process comprising:
using calcination to obtain MgO—Mg(OH)₂ nano-composites; and
aging the MgO—Mg(OH)₂ nano-composites to form the C₈H₁₀MgO₁₀.4H₂O.

13. The method in accordance with claim 12 wherein the process further comprises before calcination using Mg(OH)₂ as a precursor in electrospinning synthesis.

14. The method in accordance with claim 12 wherein using calcination to obtain MgO—Mg(OH)₂ nano-composites comprises using an aqueous solution of polyvinyl alcohol (PVA) as a polymer base to obtain the MgO—Mg(OH)₂ nano-composites.

15. The method in accordance with claim 12 wherein using calcination to obtain MgO—Mg(OH)₂ nano-composites comprises calcination at a temperature of 300° C.

16. The method in accordance with claim 12 wherein using calcination to obtain MgO—Mg(OH)₂ nano-composites comprises calcination at a temperature of 500° C.

17. The method in accordance with claim 12 wherein aging the MgO—Mg(OH)₂ nano-composites comprises ageing the MgO—Mg(OH)₂ nano-composites for a multi-month time duration.

18. The method in accordance with claim 17 wherein the multi-month time duration comprises six months.

19. The method in accordance with claim 17 wherein the multi-month time duration comprises a time duration between three months and six months.

20. Use of monoclinic magnesium malate tetrahydrate (C₈H₁₀MgO₁₀.4H₂O) for room temperature carbon dioxide adsorption.
